(19) United States
(12) Patent Application Publication
(10) Pub. No.: US 2016/0354312 A1
(43) Pub. Date: Dec. 8, 2016

(54) FORMULATIONS OF JASMONATES AND NANOCARRIERS OR MICROCARRIERS

(71) Applicant: Nanocare Technologies, Inc., New York, NY (US)

(72) Inventor: José Emilio Fehr Pereira Lopes, Sao Carlos (BR)

(21) Appl. No.: 15/170,893

(22) Filed: Jun. 1, 2016

Related U.S. Application Data
(63) Continuation of application No. 13/054,110, filed on Jan. 14, 2011, now abandoned.

(30) Foreign Application Priority Data
Jul. 15, 2008 (BR) PI0804172-5

(51) Int. Cl. A61K 9/127; A61K 31/19; A61K 31/215

(52) U.S. Cl. CPC A61K 9/127; A61K 9/1271; A61K 31/215; A61K 31/19

(57) ABSTRACT

The present disclosure provides compositions comprising a jasmonic acid derivative and a carrier, such as a liposome or cyclodextrin. Said compositions may be used for treating cancer.

### RELATED APPLICATIONS

This application is a continuation of U.S. application Ser. No. 13/054,110, filed Jan. 14, 2011, which is a national stage application, filed under 35 U.S.C. §371, of International Application No. PCT/BR2009/000151, filed May 28, 2009, which claims priority to, and the benefit of, Brazilian application No. PI0804172-5, filed Jul. 15, 2008, the entire contents of each of which are incorporated herein by reference in their entireties.

### FIELD OF INVENTION

The present invention relates to formulations of jasmonate family compounds (CA 2630666) in combination with several compounds that have the capability to form nano or micro encapsulation systems. Jasmonate Family compounds are defined as compounds with cyclopentanone type structures. They are a group of plant hormones which help to regulate plant growth and development. Jasmonates include jasmonic acid and its esters, as well many others derivatives such as for example: methyl jasmonate (MeJa). Like the related prostaglandin hormones found in mammals, the jasmonates are cyclopentanone derivatives which are derived biosynthetically from fatty acids. They are biosynthesized from linolenic acid by the octadecanoid pathway with the cyclopentanone ring. The present invention, also includes the Jasmonates with optionally artificially modified formulated structures with substitutions or inclusions of an amine molecule, and/or amide molecule, or any other substance to increase the jasmonate's effects. Modifications include transformation of the cyclopentanone ring into a cyclopentenone ring, with optional inclusions, substitutions, conjugations, additions, reductions, or any other chemical process, and optionally with any other components, of any type, inside of a nanocarrier or microcarrier, as a single molecule, or a mixture. Nanocarriers or microcarriers are compounds that can be used to form inclusion compounds. This includes the host family of cyclodextrins (CDs), more specifically the native CDs, α-, β- and γ-CD. Alternatively, we consider that it is possible for all systems that can make inclusions compounds as an opportune alternative for the CDs. Defined in these terms is a series of host molecules, particles, and aggregates as nanocarriers. In general such polymers, alternative polymers, co-polymers, liposomes, dendrimers, metallic nano spheres, mixed polymers, biopolymers, carbon structure carriers, silica structure carriers, injectable micro or nanocarriers, and nanocarriers achieve tumor-selective accumulation through enhanced permeability and retention effects. Targeting molecules such as antibodies, peptides, ligands, or nucleic acids attached to the nanocarriers further enhance their recognition and internalization by the target tissues. Nanocarriers may be activated by stimuli in the extracellular environment or intracellular environment. A nanocarrier may include nanosuspensions, nanotubes, nanowires, cationic SLN carriers, gelatin NPs carriers, PLGA NPs, PLGA nanospheres, hydrogel NPs 10 structure carriers, CPP NPs structure carriers, Polymeric micelles also known as immunomicelles, functionalized NPs, and nano crystal structure carriers. In general, this phenomenon can be characterized by the example illustrated in the reaction of formation in the scheme below:

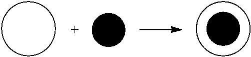

The same general reaction corresponds to equation 3 infra. The compounds formed with these formulations are characterized as efficient systems to deliver Jasmonate family members and derivatives to act macroscopically aimed at specific target cells. These target cells are, or not, characterized to be cancer cells or any other site. These inclusion compounds can act with efficient performance with a significant reduction of the toxicity of jasmonate family members and derivatives and provide chemical stabilization of the molecular structure of jasmonate family members and derivatives from hydrolysis, oxidation, and any other reaction (EP 1814894).

These formulations present herein are involved with the inclusion phenomena, the principle of host and guest interaction, resulting in a final product with a significant utility in the therapeutic field as well as many other useful properties.

### BACKGROUND OF THE INVENTION

Jasmonate compounds are characterized by the cyclopentanone ring and are already known as vegetable hormones produced and delivered in stress situations by vegetables. Qualifying as cyclopentanones, the Jasmonates are potent agents in vitro and efficiently reduce tumor cells in vivo, as demonstrated by Flescher et al (US 2002/0173470).

### SUMMARY OF THE INVENTION

In one aspect, the invention relates to pharmaceutical formulations comprising an active principle from Jasmonate's family members and its derivatives, elements, all molecules, possible formulations, and compounds into, inside, and/or at a nanocarrier or microcarrier, as hosts that can protect molecules of Jasmonate family members and all its derivatives from chemical and any other environmental reactions. Optionally, changes to its structure such as the addition or conjugation of molecules as pro-drugs and/or formulations with multiple effects for the improvement of the active principle or derivatives of Jasmonate family members and all its derivatives with, into, inside, and/or at a nanocarrier or microcarrier of any type can be useful to increase the effect. The structural changes may include possible changes at the cyclopentanone ring's structure, whether to turn it into a cyclopentenone ring, or any other possible transformations due to conjugations or any other natural or synthetic reactions on any part of the Jasmonate family members and its derivatives, inside all kinds of nanocarriers or microcarriers to obtain better performance of these molecules. More specifically the Jasmonate family members and all its derivatives and all possible formulations are used at a concentration range different than zero and one host. In one aspect, the invention relates to a process to obtain the Jasmonate family members or its derivatives inside of a nanocarrier of CDS. In one aspect, the invention relates to the Jasmonate family members, its derivatives, molecules, compounds, or products that have it carried by a nano-host or by a micro-host, composed of any possible matter and made of any material or materials. As a nano host these CDs can be native, modified, synthetic, or a mixture of CDs. CDs forming an inclusion and/or complex compound may be used as any drug, active principle element, supporting element for drug activation, in medicine, dental care, healthcare, as a supplement in food, vitamins, proteins, in veterinary, in agriculture, in industries, cosmetic, and/or other forms of host molecules which can form nanocapsules or micro capsules which are described herein as nanocarriers or microcarriers, with these compounds.

In one embodiment, the invention relates to pharmaceutical formulations where the preferred active principle includes all members from Jasmonate family members and its derivatives with, into, inside, and/or at a nanocarrier or microcarrier, which is original or synthetically created with or without substitutions and/or formed through conjugations or any different association, pure, or not, with any kind of organic element, mineral element, synthetic elements, and/or any other substance. In the case of chemical conjugations onto the Jasmonate family compounds which are carried in microcarriers or nanocarriers, the invention relates to all the organic elements, substrates, optional formulations, compounds, mineral elements, pure or impure substrates, pure or impure compounds, natural molecules with simple or complex molecular structures, synthetic molecules, semi-synthetic molecules, existent drugs, modified drugs, chemical elements used in chemotherapy, metals such as zinc, cooper, selenium, vitamins, all kinds of minerals which are single or mixtures of elements which may be used in natural or synthetic matrices, biological implants, substitute tissues, cartilage, vesicles, angiogenesis, anti-angiogenesis, artificial stents, or devices. In the case of inclusion of Jasmonate family members and derivatives in micro or nanocarriers having specialized structures to be used as drug delivery carriers, these specialized structures may be made partially or completely as, a mixture or pure which may be conjugated with all compounds, which may function as a biodegradable carrier (cationic core-shell nanoparticle), which can enclose drug molecules and allow therapeutic nucleic acids to bind onto it including, any solid or non-solid microcarrier, semi-solid or non-semi-solid nanocarriers pure or impure colloidal organic proteins which may be optionally combined with any other elements, pure or impure albumin and its derivatives, cells or other colloidal content, lipid-like peptides, natural or non-natural liposomes of any kind, ceramide liposome, ceramides, surfactant liposomes, pure or mixtures of chitosan. These specialized structures may be used in as part of an element or compound of any material and/or any methodological procedure for image-guided drug delivery. Such element or compound may include any fatty polymer such as modified or unmodified poly(ethylene glycol)-600-hydroxystearate (PEG-HS) having any activity or effect towards the P-glycoprotein such as inhibition. In one embodiment, the formulations of the invention include nanocarriers in general such as polymers, alternatively polymers, co-polymers, liposome LDE, dendrimers, metallic or non-metallic nanospheres, metallic or non-metallic microspheres, mixed polymers, biopolymers, carbon structure carriers, silica structure carriers, injectable micro or nanocarriers, tumor selective nanocarriers, and nanocarriers targeting by landscape or any other ataxia effect, carriers which target molecules such as antibodies and peptides, ligands which may produce an effect, including nucleic acids attached to the nanocarriers which further enhance their recognition and internalization by the target tissues, microcarriers or nanocarriers activated by stimuli which may or may not be in the extracellular or intracellular environment, micro or nanosuspensions, carriers having ligand-receptor effects, long-circulating, optionally PEGylated pharmaceutical carriers, short-circulating pharmaceutical carriers, and bimetallic, pure or impure nanorods that can simultaneously bind compacted DNA plasmid and targeting ligands in a spatially defined manner. In one embodiment, the formulations of the invention include carriers that may have some effect on membrane-destabilizing lipid components, carriers with anionic polymers, carriers functionalized with proteins and peptides, or any other element, that demonstrate a unique ability to penetrate into cells (“protein transduction” phenomenon) and therefore may serve as a “transport” through the cell membrane, and/or the ability to penetrate into virus or fungus. In one embodiment, the formulations of the invention include carriers functionalized with polymeric components with pH sensitive (pH-cleavable), or linear bis polyethylene glycol (PEG) polymer with branched or un-branched spacers. In one embodiment, the formulations of the invention include carriers such as polyamidoamine (PAMAM) containing an optional G4 hydroxyl terminated dendrimer, conventional and PEGylated liposomes in multifunctional nanocarriers or microcarriers, and any other carrier with controlled properties with the requirement such as conjugation of proteins, peptides, polymers, cell-penetrating moieties, reporter groups and other functional ligands to the carrier surface with properties such as non-covalent attachment via the hydrophobic adsorption with intrinsic or specially inserted hydrophobic groups in the ligands to be attached onto or into the surface of the nanocarrier. For example, the attachment is performed chemically, or via the interaction of reactive groups generated on the carrier surface and certain groups in the molecule to be attached. In one embodiment, the formulations of the invention include peptide nanotubes, and microcarriers or nanocarriers which are made entirely or in part of hyaluronan polymers which may or may not be of the intralymphatic. In one embodiment, the formulations of the invention include peptide nanovesicles, lipid nanotubes for simultaneous or sequential delivery of resistance modulators (e.g., with P-glycoprotein substrates). In one embodiment, the formulations of the invention include agents that regulate intracellular pH, agents that lower the apoptotic threshold (e.g., with ceramide), or in combination with energy delivery (e.g., sound, heat, and light) to enhance the effectiveness of anticancer agents in refractory tumors or any other disorders. In one embodiment, the formulations of the invention include nanotubes, nanowires, microwires, cationic solid lipid microparticle or nanoparticle carriers, gelatin nanoparticle carriers, carriers made of components derived from egg, polylactic glycolic acid nanoparticles, polylactic glycolic acid nanospheres or microspheres, hydrogel microparticle or nanoparticle structure carriers, copolymerized peptide microparticle or nanoparticle structure carriers, polymeric micelles known as immunomicelles, functionalized nanoparticles, nanocrystal structure carriers, or magnetic nanoparticles able to be attached to a photosensitive linker connected to any kind of drug, molecule, or elements that may or may not be conjugated participating through any physical or chemical reaction, to the magnetic nano-assembly or carriers that transform itself, liberating factors or properties or undergoing any kind of morphological transformation due to the actions of molecules or any other kind of signaling, infra, extra body, into the body, and/or at the body, of intracellular or extracellular Jasmonate family compounds in solution.

In one embodiment, the invention relates to pharmaceutical formulations as described herein where CD is used as nanocarriers or microcarriers and is optionally 2-,4-,6-trimethyl-CD, heptakis-6-sulphate-CD, hydroxypropyl-CD, large ring CDs, with a range of units between 8 and 26 glucose units or chains, or polymers, which may or may not be associated with the smallest CDs inside of the cavity of large ring CDs forming double system inclusion compounds. In one embodiment, formulations of the invention include CDs that can be synthetic that are linked with any kind of organic, mineral elements, pure or impure biological elements which are optionally mixtures, or pro drugs associated with DNA or RNA type molecules and the Jasmonate's family members and its derivatives which are modified by any possible inclusion, conjugations, and/or any optional change in molecular structure, within CDS that that are changed chemically, morphologically, pure, impure, conjugated, non-conjugated to become electrically charged, magnified, photo reactive, light emissive structures, PH reactive, chemically reactive, radiation sensitive and reactive, useful for computerized controlled drug delivery, computerized structures for molecular guidance, able to be controlled at a distance and associated at the nanocarrier or microcarrier, or any substance such as chemotherapy agents, antibiotic, antifungal, anti-inflammatory, corticoid, protein, carbohydrates, hormones, lipids, cosmetic, agriculture issue, or any industrial uses.

In another embodiment, the invention relates to pharmaceutical formulations, as described herein, where a list of host molecules other than CDs are used as carriers, the list comprising liposome, different kinds of dendrimers as a drug carrier independent of its formations and built structures, as an example: PAMAM/MTX and PAMAM-PEG/MTX, or optionally other dendrimer types t, supramolecular nanocarriers for gene and siRNA delivery, semi-permeable polymer nanocarriers for enzyme therapies, all kinds of polymers, optionally biopolymers which are non-synthetic or synthetic, pure or impure, optionally made as a singular element, or composed in chains with more than one type of polymer and biopolymer which is optionally a mixture, with other kinds of nanocarriers or microcarriers, including, nanocarriers and microcarriers having in their structures any kind of elements, pure, as a mixture, natural, modified, semi-synthetic, or synthetic, which are made of heteromorphism structures or as homomorphism structures, which include elements as part of its structure or as the carrier itself such as biological structures, blood elements as carriers or any other use, biological or colloidal substances which are natural, mixtures, synthetic, or semi-synthetic, pure or impure, natural or unnatural proteins, glycoproteins, enzymes, lipoglycoproteins, antibodies, cells, vegetal cells, organic fluids, elements, pure or not, and/or natural or not and/or animal extracts, plants extracts, vegetal elements and compounds, fungus substrate, virus substrate, bacteria substrate, natural compounds and its substrates, or crystals, carbon structure, gold or any metal, nanospheres or microspheres which is a mixture or pure, pure or impure liposome which is optionally, pure or not, and/or modified, a mixture, or synthetic, unique lamellar vesicles, multi lamellar vesicles, which are alternatively administered with polymers that are artificial, pure or impure LDE, pure or impure, synthetic or non-synthetic polymeric structures, dendrimer structures, talosphere structures, nanosphere structures, metal structures, mechanical structures, computerized structures, electronic structures, magnetic structures which are optionally biologically produced, biological sensitive structures, chemical sensitive structures, radiation sensitive structure, thermal sensitive structures, electric sensitive structure biopolymers which are optionally a mixture, mixtures of lipo polymers, or organic substances able to become a microcarrier or a nanocarrier of the Jasmonates family member compounds. In one embodiment, formulations of the invention include mixtures of pure or impure, synthetic or non-synthetic elements and nanoparticles made of pure or mixtures of mineral elements such as pure or impure, synthetic or non-synthetic silicon, or carbon, pure or a mixture of dendrimers combined with other elements, microspheres or microcarriers in general or polymeric CDs, CDs nanocontainers (carcerands), pure or impure, synthetic or non-synthetic crystal or metal nanostructures, biological structures, pure or a mixture of mineral structures, pure or a mixture of synthetic structures modified at the surface within inductive substances with the following effects: blocking, conduction, producing, patenting, especially patenting biological tropism, analogues, competitive, synergic, superficially modified, with multi inductive substances, interactive, lighting nanoparticles, nanoparticles derived from silica, silicon, evaporating particles, heat sensitive particles, gas or any other chemically liberated particles, microparticles or nanoparticles formed to target specific receptors in vegetable or animal derivatives, optical or perfusion fluids with the use of preservation of organic tissues, transplant, or any other tissue use. In one embodiment, formulations of the invention include nanocarrier systems to enhance the bioavailability of drugs at the disease site. Nanocarrier systems incorporated with any stimuli-responsive property optionally developing as it delivers due to physical or chemical reactions (e.g., pH, temperature, or redox potential, Temperature, pH, and hypoxia are examples of “triggers” at the diseased site that could be exploited with stimuli-responsive nanocarriers), or any other response, involving itself only or other molecules or substances, including stimuli-responsive nanocarrier systems for drug and gene delivery, any nanocarrier able to perform the effect of gene delivery, carriers made that can respond to biological stimuli, or microcarriers or nanocarriers able to change its morphological structure during its delivering procedure, before it, or after it such as the optimization of nanocarrier size and surface charge modulation.

In one embodiment, the present invention relates to pharmaceutical formulations, as described herein, and use thereof in a chemical therapeutic sense to decrease the side effects, associated with any kind of treatment as a single therapy or mixture of therapies with its actions on the interference in cell growth, inhibited signal, interference in cellular apoptosis. In one embodiment, the present invention relates to the effect of the molecules of the invention to be used, partially, or in its totality as an ingredient, element, or part of any compound, with its action towards cells, such as in the effect on the limitless replicative potential of a cell, sustained angiogenesis by a cell in favor of the effect of anti-angiogenesis, controlling cellular tissue invasions and/or cancer metastasis, or any effect on the tumor development or regression. In one embodiment, the present invention relates to the use of the molecule of the invention in any other therapy such as: biotherapy, chemotherapy, radiotherapy, angiogenic or antiangiogenic therapy, genetic therapy, surgeries, bio molecular manipulation as part of any therapy, surgery of any kind, plastic surgery, photo dynamic therapy procedures, dental procedures, cosmetic surgery or any cosmetic application, surgery for healing scars and wounds, with any kind of laser and light therapy. In one embodiment, the present invention relates to the use of the molecule of the invention as a drug for AIDS, or any virus disease, bacterial disease, body dysfunctions, as a component to be used in an anti-inflammatory agent, pain reliever, anti-septic solutions, anti-fungus solutions, anti-virus solutions, anti-bacterial solutions, or part of any chemical purpose. In one embodiment, the present invention relates to the use of the molecule of the invention as a drug for fungal disease, auto immune disease, dystrophies, mental diseases, depression associated with neurological effects, antidote for poisoning or any other dangerous or harmful afflictions, inducer or activator of mechanisms for bioresults, anti-smoking drug, vaccines, any other kind of intracellular or extracellular activator, organic molecule such as inhibition factors, or angiogenic or antiangiogenic therapies, which are single or in association with therapy in animals or in humans in the sense to decrease the side effects of any kind. Since these drugs are encapsulated in the case of the present invention, the Jasmonate family members and its derivatives which are oily and demonstrate low solubility, can be turned into a soluble molecule which allows better pharmacokinetics to produce products that can be made for oral, intra dermal, dermal, surgical, topical such as epidermal and mucosal uses, skin appendages, endoscopic procedures as well as intra orifice use, mechanical or guided, laparoscopic procedures, parenteral nutrition, intra brain procedures, lumbar punctures, cosmetic procedures, sub dermal procedures, any tissue procedures, transdermal, spine punctures or procedures, intramuscular, inhalation, ocular, dental, endogenous administrations, sublingual, subcutaneous, rectal use, or any other uses into mucosal, and/or at, and/or inside.

### BRIEF DESCRIPTION OF THE FIGURES

FIG. 1 shows the up and side view of a) α-, b) β- and c)-γ-CDs.

FIG. 2 shows the up and side view of molecular structures between Jasmonic acid and a) α-, b) β- and c)-γ-CDs.

FIG. 3 shows the up and side view of molecular structures between Methyl Jasmonate and a) α-, b) β- and c)-γ-CDs.

FIG. 4 shows the molecular structure of PAMAM type dendrimers.

FIG. 5 shows the experimental results for dendrimer association with methyl Jasmonate.

FIG. 6 shows the experimental results for β-CD association with methyl Jasmonate.

### DETAIL DESCRIPTION OF INVENTION

The Jasmonate family is defined as: methyl jasmonate, jasmonic acid, 7-iso-jasmonic acid, 9,10-dihydrojasmonic acid, 2,3-didehydrojasmonic acid, 3,4-didehydrojasmonic acid, 3,7-didehydrojasmonic acid, 4,5-didehydrojasmonic acid, 4,5-didehydro-7-isojasmonic acid, cucurbic acid, 6-epi-cucurbic acid lactones, 12-hydroxyjasmonic acid, 12-hydroxyjasmonic acid lactones, 11-hydroxyjasmonic acid, 8-hydroxyjasmonic acid, homo-jasmonic acid, dihomojasmonic acid, 11-hydroxy-dihomojasmonic acid, 8-hydroxy-dihomojasmonic acid, tuberonic acid, tuberonic acid-O-β-glucopyranoside, 5,6-didehydrojasmonic acid, 6,7-didehydrojasmonic acid, 7,8-didehydrojasmonic acid, cis-jasmone, dihydrojasmone, methylhydrojasmone, conjugated jasmonic acids with amino acids and esters with lower range alkyl chains linked with all possible substituents and stereoisomers. Optionally, the Jasmonate family compounds can be associated as a prodrug through an amide and/or esters chains, and/or with other linkages. The definition of prodrug is concerned with the structures of compounds after being metabolized inside the chemical environment of an animal body. Reactions such as hydrolysis or oxidation/reduction, or any metabolic or catabolic organics reactions, sometimes break a specific chain and produce two or more others with metabolic, or catabolic action as medical drugs and others. Specifically in this invention this kind of combination could be useful to obtain a better performance of the final product. In many instances, Jasmonate family members as well as its derivatives are oily and demonstrate low solubility. Since these drugs are encapsulated in the case of the present invention, Jasmonate family members and derivatives can be turned into a soluble molecule. This will allow for better pharmacokinetics and provides products that can be made as/for oral, intradermal, dermal, surgery, topical such as epidermic and mucosas uses, skin appendages, endoscopic procedures as well intra orifice uses, mechanical or guided, laparoscopic procedures, parenteral nutrition, intra brain procedures, lumbar punctures, cosmetic procedures, sub dermal procedures, any tissue procedures, transdermal, spine punctures or procedures, intramuscular, inhalation, ocular, dental, as endogenous administrations, sublingual, subcutaneous, rectal use, or any other uses via mucosas. Also the nanocarried or microcarried elements of the Jasmonate family can be modified in its structure to improve it actions towards innumerous different aims. It can be changed in its cyclopentanone ring, increasing or making substitutions, turning it to cyclopentenone, or adding innumerous other elements to its structure to improve its effects. The Jasmonates family elements can also be used to formulate new compounds to be included with it inside of the nano or microcarriers.

The designation microparticles or nanoparticles applies to the controlled delivery of molecules that are enlarged and refers to all types of different structures that are able to form nano spheres, nano capsules, microspheres and microcapsule carriers that can carry the molecules referred to in this invention. Designated are nanosphere systems where the active principle is homogeneously dispersed or soluble inside of the polymeric matrix. In this sense the system obtained is unique and it is impossible to distinguish between the host and guest molecules. Otherwise, nanocapsules are systems where it is possible to identify the two phase compounds. In these compounds the active principle is able to be identified and thus it is possible to distinguish a difference between the two systems, host and guest. Sometimes the two systems are made in different phases, solid and liquid phases. In these cases the substances are involved with a polymeric matrix, usually one membrane, isolated to the nucleus.

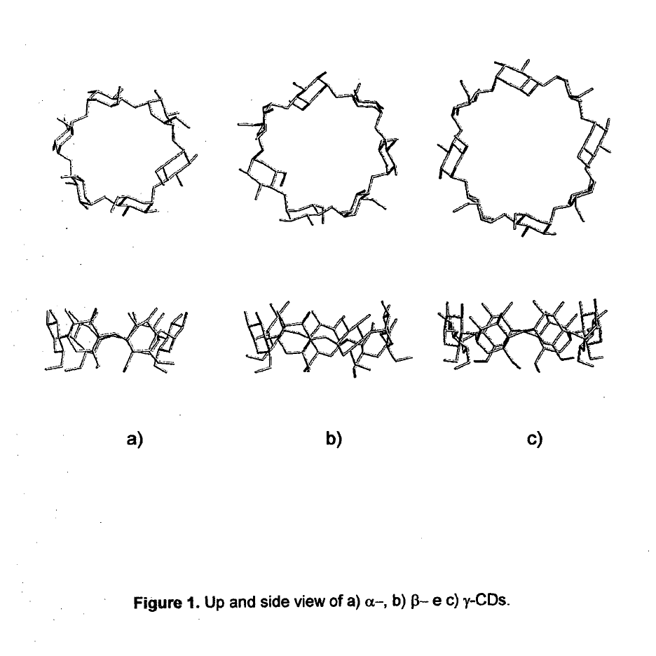

Cyclodextrins (CDs) are cyclic oligosaccharides formed by D-L(+)-Glucose units linked by α-1,4-C—O—C chains. CDs are obtained by the enzymatic degradation of starch with the CGTase glucotransferase. The native CDs are defined by the number of glucose units, α-, β- and γ-CDs obtained with 6, 7 and 8 glucose units. FIG. 1 shows the molecular structure of these natives CDs.

From a structural point of view the CDs are cone shaped molecules. In the molecular structure of CDs there are two sides, hydrophobic and hydrophilic. Inside of the cavity of CDs the hydrophobic character is predominant. This characteristic is important to guide the guest molecule to locate spontaneously inside the cavity. This principle is called inclusion compound formation phenomena. From a thermodynamic aspect, the spontaneous formation rule states that the lower energy state of a system is the most probable one to occur. This phenomenon is observed in the classic experimental formation of micelles. The inclusion compounds formation occurs because it is the lower energy state between the molecules due to the hydrophobic effect. Otherwise, the hydrophilic part, which is outside the CD cavity, contributes to the stability of the inclusion compounds formed. This phenomenon provides the possibility for the use of low solubility or highly toxic molecules as the active principle of pharmaceutical products. Nowadays this type of technology is already established and useful in pharmaceutical industries and other technical applications.

The CDs can form inclusion compounds with a notable number of guest molecules and are used in different applications in pharmaceutical, food and cosmetic products. The molecular encapsulation shows practical advantages for new formulations of known products. Many known molecules that were neglected by the industry in the past can be studied in new formulations with these microcarriers or nanocarriers. It is very probable that a new product remains for a large number of applications. CDs have been mentioned, but there are many other elements and molecules, natural, synthetic, semi-synthetic and mixtures that have been projected to form nanocarriers to be able to carry drugs and many others substances inside of it. Each one has a specific aim or property to show its effect. In one aspect the invention relates to all the micro and nanocarriers made of these elements and compounds; molecules such as microcarriers and nanocarriers made by any chemical reaction, using natural or non-natural elements, semi synthetic or non-semi-synthetic materials, or synthetic or non-synthetic materials. In one aspect the invention relates to compounds will create new formulations of micro or nano capsules or carriers within Jasmonate family members. The Jasmonate family members may be in its pure form, a mixture, or conjugated with any other drug or drugs or substance which may increases its effects. Jasmonate family members may have its structure modified in any part along its structure, or be pure or associated with any other substance or derivative from plant or any animal, including elements and substrates from microorganisms, drugs, and any active principle to reduce the toxicology of the drug, and active molecules having the Jasmonate as a co-factor or a co-helper. The Jasmonate family members may also be associated with another drug or organic molecules, organic substrates, organic elements, pure, mixture or conjugate, mineral elements, synthetic, or semi-synthetic to improve its function as co-helper, or increasing the active principle of the Jasmonate's family members or elements derived from it. To be useful, as an example: In the medical field, in all areas, in order to make the formulated molecule reach the aimed target, one may use any possible components to build up the micro, nano capsules, carriers, or biomarkers that will increase the action of the Jasmonate's elements and family or its derivative members. The Jasmonate family members or derivative members may be conjugated to any other molecule that may associate with any other elements and molecules including; proteins, glycoprotein, lipids, organic elements, and mineral elements. These conjugates may result in single or multi structured compounds or molecules to be used in many industrial fields, including: chemical, physical, mechanical, structural, agricultural, veterinarian, and cosmetics for the production of elements to be used in the manufacturing of products of any kind of industrial field. The present invention relates to all the Jasmonate family members, or derivatives from it, as well as all the possible molecules formed with it, whether in its pure form, modified, conjugated, mixture, complexed, or any other molecule associated with it. These molecules may be included in elements or molecules able to form micro or nanocarrier compounds with properties to demonstrate better effects of Jasmonate family members, or derivatives, with all the possible molecules associated with it including any element that has one or more properties such as, magnet properties, electrical properties, chemical properties, photo sensibility properties, morphologic properties, bio acceptance properties, non rejection properties, physiological properties, body response properties, protection properties, dental properties, organic, non-organic, organic ataxia effect, all types of ataxia properties, radiation properties, remote controlled guidance properties to the micro, and or, nano host, fluorescence properties, and thermal properties. Projected new structures for better carrier compounds also includes modified morphological surface polymers that are (1) added or conjugated with pure or synthetic or modified, or a mixture with organic components, (2) added or conjugated with pure or synthetic or modified, or a mixture with lipid components, (3) added or conjugated with pure or synthetic or modified, or a mixture with mineral components, (4) added or conjugated with pure or synthetic or modified, or a mixture with metal components, (5) added or conjugated with pure or synthetic or modified, or a mixture with carbon components, (6) added or conjugated with pure or synthetic or modified, or a mixture of all elements above with computerized components, (7) added or conjugated with pure or synthetic or modified, or a mixture of cellular, micro or nanocarriers made using parts, added at, into, or with, bacteria, or mixtures of components, (8) added or conjugated with pure or synthetic or modified, or a mixture of molecule components, virus, or mixtures of components, (9) added or conjugated with pure or synthetic or modified, or mixtures of molecule components, fungus, or mixtures of components, (10) added or conjugated with pure or synthetic or modified, or mixtures of molecule components, body solids, elements, or mixtures of components, (11) added or conjugated with pure or synthetic or modified, or mixtures of molecule components, or body's fluids, lymph and blood elements, or mixtures of components, (12) added or conjugated with pure or synthetic or modified, or mixtures of molecule components.

Other classes of molecules that can interact forming structures like inclusions as micro and nanocarriers contemplated in the present invention are block co-polymers such as Pluronic®, a relatively hydrophilic polymer and poly-ε-caprolactone obtained by the scission of the ε-caprolactone ring in the presence of PEO-PPO-PEO using stannous octoate as a catalyst. (Drumond W. S.; Wang, S. H., 2004). There are also other biopolymers that are contemplated in the present invention that can be used as structural carriers to provide a better effect of the molecule in its expected aim.

The present invention can be a multiple formulation of Jasmonate family members, other molecules, and other possible Jasmonate family member compound derivatives, that may be associated with a large number of different elements to form stable inclusion compounds including nanocarriers or microcarriers, nanoemulsions, and others that can be used to produce all the possibilities described above. The present invention relates to obtaining an improvement of the delivery of Jasmonates family members and its derivatives, and possible derived compounds, which may be pure, modified, a mixtures, or conjugated, and various formulations, within, at, and/or with micro and nanocarriers.

The invention also refers to the micro and nano particles within, with, and/or at Jasmonate derived compounds, which may be pure, modified, a mixture, or, conjugated, and various formulations within, at, and/or with micro and nanocarriers which are used to interact with any inhibitory drugs used to treat hypoxic condition in normal or cancerous tissue. In one aspect the invention relates to the interaction of members of the Jasmonate family or, derivative members with other possible molecules which may be included in or form inclusion compounds in micro particles or nano particles to function as carriers. In one aspect the invention relates to the interaction of members of the Jasmonate family or derivative members as a pure substance or a mixture with members of molecules that can produce an effect in fundamental biochemical pathways which may include:—DNA synthesis, transcription, translation, gene regulation and energy production which are first initiated under conditions of anoxia, such as anaerobic glycolysis. That functions best in hypoxia. It is these pathways that are up-regulated in the hypoxic cancer cell. The effect of the molecule of the invention with its action on the trans-activating domain (TAD) amino acid compositions, which are either essential for Trans activation or are the most abundant amino acids in TAD are used for generation of TADs groups. The Trans activation by transcription factor Gal4 was found to be provided by acidic amino acids and therefore Gal4 is referred to as the transcription factor with acidic activation domain. Gcn4 is referred to the transcription factors with hydrophobic activation domain.

A nine-amino-acid trans activation domain (9aaTAD) defines a novel domain common to a large super family of eukaryotic transcription factors represented by Gal4, Oaf1, Leu3, Rtg3, Pho4, GIn3, Gcn4 in yeast and by p53, NFAT, NF-KB, any other gene translator factor, nuclear or not, 10 and VP16 in mammals.

The effect of the molecule of the invention with its action on the DNA, RNA of cells, mRNAs, clones, oncogenesis, proto oncogenesis, pro apoptosis of cellular groups, anti apoptosis of cellular groups, anti or pro cellular fatigue, senescence, virus, fungus, or bacteria.

In one aspect the invention relates to the effect of the molecule of the invention with any mentioned action involving immunological cells and its members, and its substrates. In one aspect the invention relates to the effect of the molecule of the invention, including all previously described actions involving intracellular participation, and participation in the actions including: phosphorylation process, glycolysis, anaerobic or aerobic energetic metabolic processes involving any mitochondria processor the electron transport chain, suppression or activation of the activity of a group of cysteine proteases called caspases, apoptosis inducing factor, Fas receptor (FasR), any group involved with death inducing signaling complex_(DISC), any function with adaptor molecule FADD, death effector domain (DED) near its amino terminus-which facilitates binding to the DED of FADD-like ICE (FLICE) which is more commonly referred to as caspase-8, proteolytic cleavage, and the effect of the invented molecule, pure or not, conjugated or not, with its actions on, as, or into the caspase-8 catalyzed cleavage of the pro-apoptotic BH3-only protein Bid into its truncated form, tBid. In one aspect the invention relates to any of the micro or nano molecules described herein are involved in any circumstances or any mode with BH-3 only members of the Bcl-2 family which engage exclusively anti-apoptotic members of the family (Bcl-2, Bcl-xL). In one aspect the invention relates to any action that may allow the invented molecule to allow or increase Bak and Bax translocation to the outer mitochondrial membrane, thus permeabilizing it and facilitating release of pro-apoptotic proteins such as cytochrome c and Smac/DIABLO, an antagonist of inhibitors of apoptosis proteins (IAPs).

In one aspect the invention relates to the effect a molecule, pure or not, conjugated or not, with its actions on or as involved in any circumstances or any mode with cells of dubbed Type 1 cells that are characterized by the inability of anti-apoptotic members of the Bcl-2 family (namely Bcl-2 and Bcl-xL) to protect from Fas-mediated apoptosis.

In one aspect the invention relates to a molecule which is involved in any circumstances or any mode with the characterized Type 1 cells including H9, CH1, SKW6.4 and SW480, all of which are lymphocyte lineages except the latter, which is a colon adenocarcinoma lineage.

In one aspect the invention relates to a molecule, pure or not, conjugated or not, with actions including increasing functions, decreasing functions, co-helpers, up regulating, down regulating, acting as an inhibitory factor, activating factor, participating in any metabolic and catabolic cellular action, alone or conjugated, and participating in any action as a cellular co-factor, at or with all of the following intracellular sites: STATs, CR, MAPK, SV 40 promoter-1 (SP1), E26 (Ets), NF-AT,GATA-3, JNKs, Rel A, Rel B, IkBs and all forms of NF-KB, all proteins that belong to the NF-KB complex, AP-1, as agonists or antagonists of the nuclear receptors, all the PPars, to react with lipo-poly saccharide molecules, as substrate or agent, to interact with any agent involved in the inflammatory process, as all cytokines and ICAM-1, VCAM-1, with COX-1, COX-2, as agonists or antagonists of all prostaglandins, leukotrienes, pure or not, thromboxanes, with all chemo cytokines, monoclonal cells carrying cancer cells, Dendritic cells, T cells, B cells, CD1, CD4, CD8 and any subclass of it, memory cells, natural killers, and all blood cells, blood proteins, pure or not, conjugated or not, natural or artificial cloned cells.

The action of the molecule of the invention, pure or not, conjugated or not, with actions including increasing functions, decreasing functions, co-helpers, up regulating, down regulating, acting as an inhibitory factor, or activating factor of the VEGFs, all the growth factors, all its cellular receptors, the cellular receptors of all cytokines, all metalloproteinases, αFGF, βFGF, TK cell membrane receptors, G protein mediated cell membrane receptors, any other cellular receptor, cell substrate inducers, biomolecule inducers, immunological inducers, and direct or indirect action with PDGF, HIF, polypeptide growth factors, TGFα and TGFβ, (TGFβ exists in three known subtypes in humans, TGFβ1, TGFβ2, and TGF(β3), Interferons, Ils, Tumor necrosis factor (TNF, cachexin or cachectin and formally known as tumor necrosis factor-alpha), Lymphotoxin (also known as tumor necrosis factor-beta) and any biological molecule production. All cytokines bind at the following targets: Immunoglobulin (lg) superfamily, which are ubiquitously present throughout several cells and tissues of the vertebrate body, and share structural homology with immunoglobulins (antibodies), cell adhesion molecules, and even some cytokines. Examples include IL-1 receptor types.

The effect of the molecule of the invention with any action described herein involving in any kind, pathway or bonding with Haemopoietic Growth Factor (type 1) family, whose members have certain conserved motifs in their extracellular amino-acid domain. The 1L-2 receptor belongs to this chain, whose γ-chain (common to several other cytokines) deficiency is directly responsible for the x-linked form of Severe Combined Immunodeficiency (X-SCID), Interferon (type 2) family, whose members are receptors for IFN β and γ. Tumor necrosis factors (TNF) (type 3) family, whose members share a cysteine-rich common extracellular binding domain, and includes several other non-cytokine ligands like CD40, CD27 and CD30, besides the ligands on which the family is named (TNF).

The effect of the molecule of the invention with any action described herein involving in any kind, pathway or bonding with seven, and/or/any transmembrane helix family, the ubiquitous receptor type of the animal kingdom. All G-protein coupled receptors (for hormones and/or neurotransmitters) belong to this family. Chemokine receptors, two of which act as binding proteins for HIV (CXCR4 and CCR5), also belong to this family. The effect of the molecule of the invention with any action described herein involving in any kind, pathway or bonding with all elements belonging and involved at the formation or being produced directly or indirectly in the immunological response, involving molecules, and complex substrates of immature and mature dendritic cells.

### Physics and Chemical Characterization of the Invention

As an example, a complete study for natural CDs and Methyl Jasmonate and Jasmonate Acid was performed, which includes all the nano capsules and micro capsule's inclusion or process of using the Jasmonates family members, and its derivatives carried with it. One of the most popular methods is the characterization of the inclusion compound's formation with theoretical Qualitative Structure Analyses Relationship (QSAR) applied with HYPERCHEM software using semi-empirical approach. In this sense QSAR was used to estimate the stability of the inclusion compounds formed between jasmonic acid and methyl Jasmonate inside of the native CDs. The calculation was performed with AM1 semi-empiric method using Polak-Rabiere conjugated gradient with rms of 0.1 kcal. (angstron·mol)⁻¹.

Assuming ΔG=ΔH we can write the equilibrium constant of the reaction of formation of the inclusion compounds as a function of entropic term:

ΔG=−RTInK [1]

Where

ΔG=−RTInK [2]

Qualitatively the measurement of ΔH (=E_{binding}) reflects the lowest of the total energy of the system when inclusion compound formation occurs.

Therefore, the stability of the reaction represented by E_{binding} can be estimated from the total energy of the all individual components of the reaction of formation of the inclusion compounds, as:

S+CD S:CD [3]

In this way we can write:

ΔH=ΔH_{RS:CD}−(ΔH_{IS}+ΔH_{fCD}) [4]

Table 1 shows the results of the calculation for the inclusion compound's formation between methyl Jasmonate, jasmonic acid and the natives CDs.

TABLE 1
The result of the ΔH of stabilization.

| | Ebinding (Kcal · mo1−1) |
|---|---|
| Jasmonic acid-α-CD | −9.63 |
| Jasmonic acid-β-CD | −19.64 |
| Jasmonic acid-γ-CD | −2.02 |
| Methyl jasmonate-α-CD | 8.44 |
| Methyl jasmonate-β-CD | −13.35 |
| Methyl jasmonate-γ-CD | −18.23 |

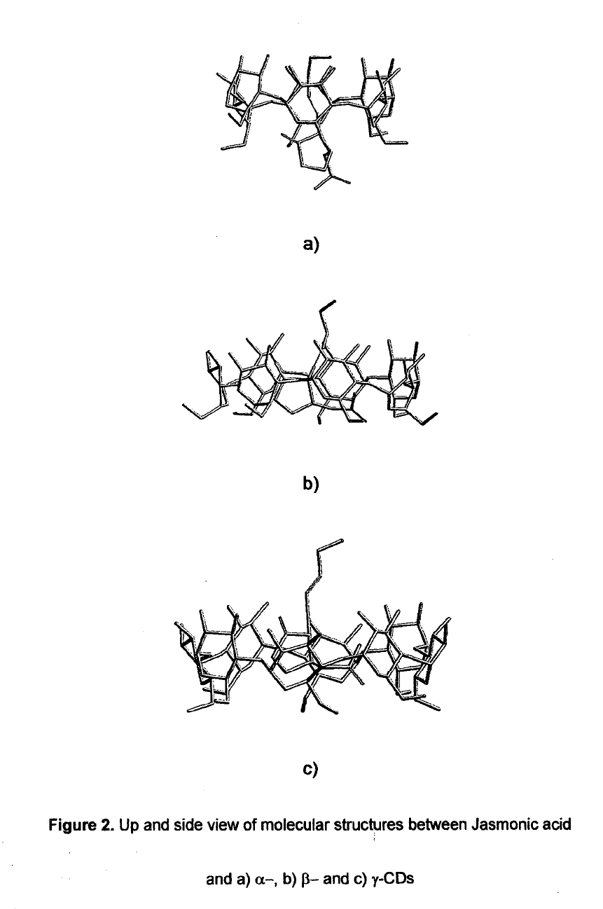

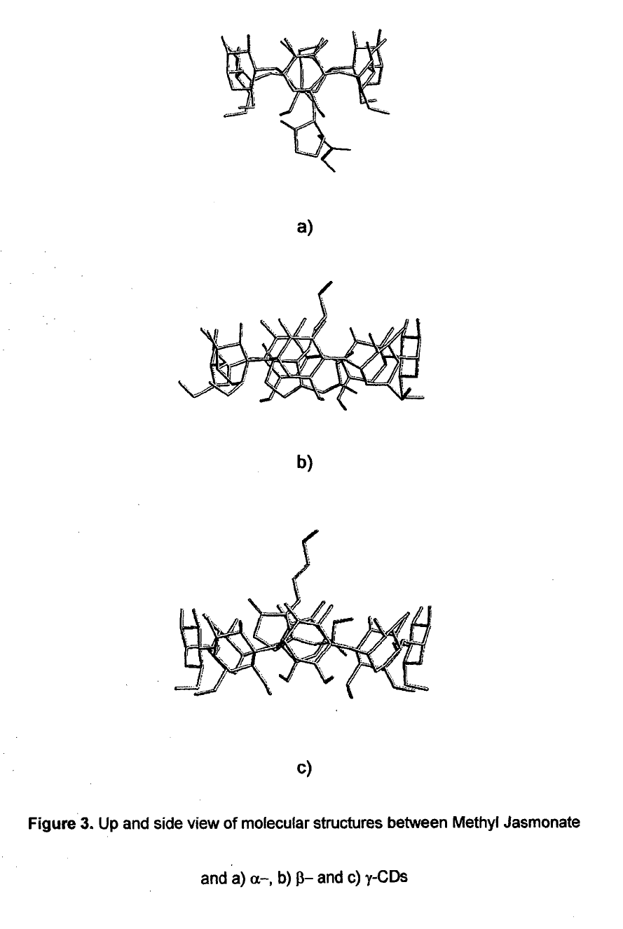

Table 1 lists some significant results. First, with the exception of the complex between α-CD and methyl Jasmonate, all the inclusion compounds between Jasmonic acid and methyl jasmonate with native CDs are stable. Second, in both cases the complex with β-CD is most stable. With Jasmonic acid α-CD is more stable than γ-CD and with methyl Jasmonate it is the reverse. FIG. 2 shows the molecular structure of the lower energy structure for Jasmonic acid and FIG. 3 shows the results for methyl Jasmonate. Note that in the case of α-CD the guest molecules in both cases are a little outside of the cavity than the other CDs.

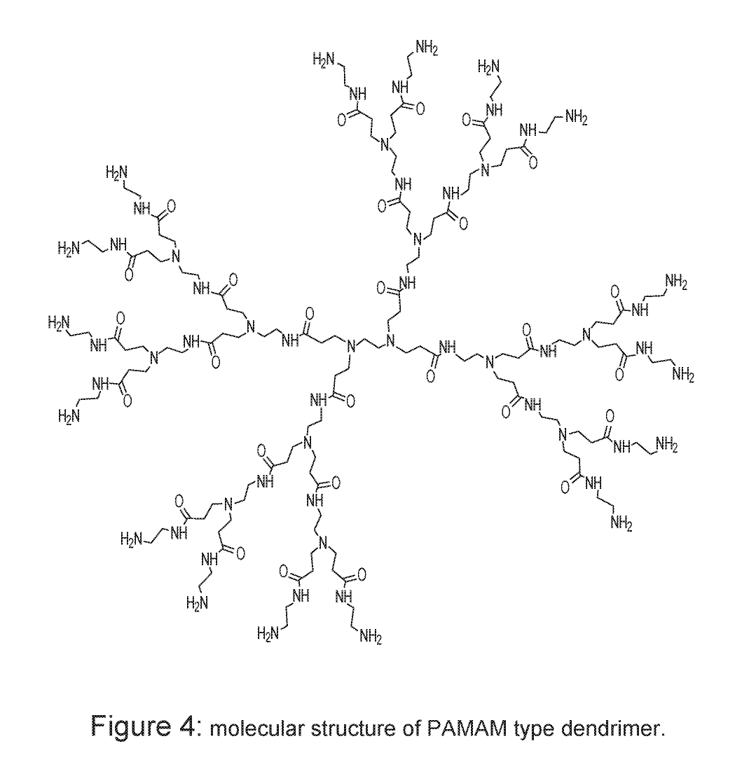

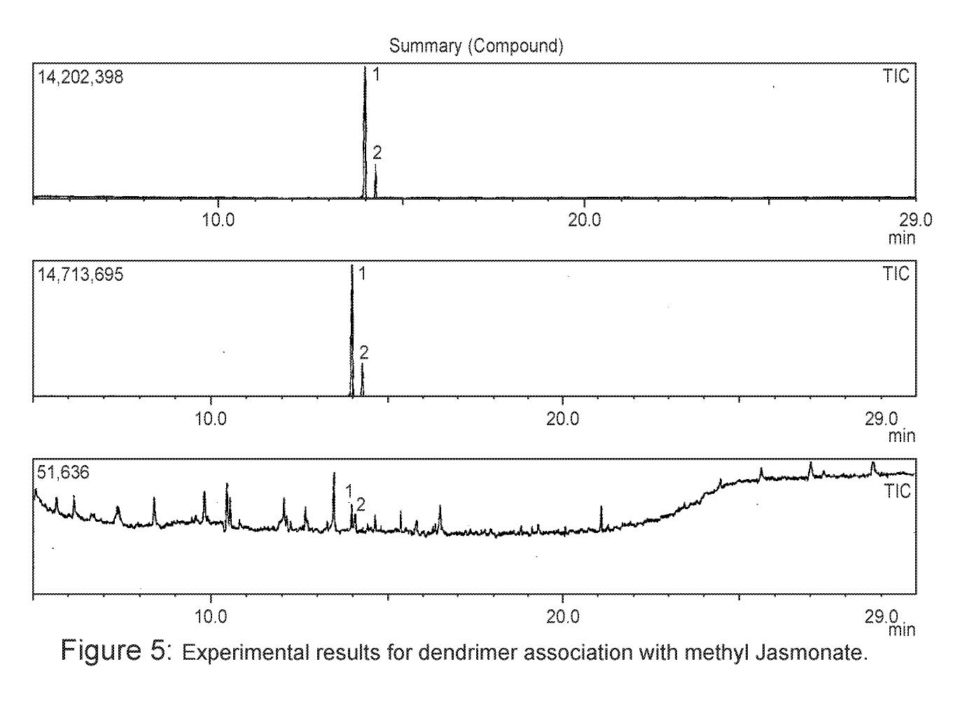

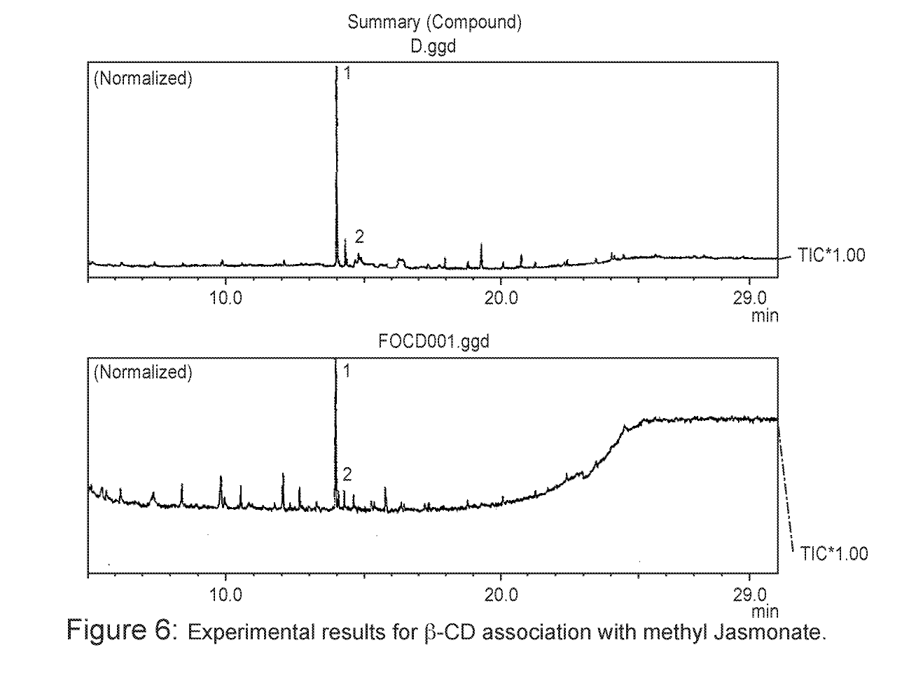

To prove the universal correlation with other nanocarriers, experimental analyses with GC/MS equipment for dendrimers and CDs was used to compare the analytic computation. The class of dendrimers used was PAMAM. FIG. 4 shows the general structure of PAMAM dendrimers. FIG. 5 shows the results for dendrimers and FIG. 6 shows the results for β-CD, both with methyl Jasmonate.

The experimental data was obtained using a GC with a column temperature of 50° C., the injection temperature of 250° C., Flow control mode linear, total flow 50.0 mL/min., and column flow of 1.70 mL/min. The percentage relative to the incorporation of methyl jasmonate inside β-CD was 98-99% and for dendrimer PAMAM it was more than 95%. Comparing peak 1 with 2, peak 2 is methyl Jasmonate alone and peak 1 is the inclusion compounds formed. In both cases peak 1 is clearly different and proves the molecular association.

### Examples of the Preparation of Invention

Inclusion compounds between the Jasmonates and nanocarriers or microcarriers can be prepared mixing a concentration between zero and 1 M with a molar equivalent of 20 in proportion to the host molecule. The preparative procedure involves mixing the Jasmonate family compound in a solution in water or other solution with pharmaceutical salts. The resulting solution is stirred until total dissolution of the components in the solvent. Usually the time of mixing is several hours until the mixture reaches thermodynamic equilibrium (Rajewski & Stella, 1996).

### PATENT REFERENCES

CA 2,630,666
EP 1814894
US 2002/017347

### NON PATENT REFERENCES

Drumond W. S.; Wang, S. H. “Sintese e Caracterizacao do copolimero Poll acid latico-B-Glicol Etilenico” Polimeros: Ciencia e Tecnologia, 14,n 2, p. 74-79, 2004
Rajewski R A, Stella V J. Pharmaceutical applications of cyclodextrins. 2. In vivo drug delivery. J Pharm Sci 1996; 85(11):1142-69.

What is claimed is:

1. A pharmaceutical formulation comprising an active principle selected from methyl jasmonate, jasmonic acid, 7-iso-jasmonic acid, 2,3-didehydrojasmonic acid, 3,4-didehydrojasmonic acid, 3,7-didehydrojasmonic acid, 4,5-didehydrojasmonic acid, 4,5-didehydro-7-isojasmonic acid, cucurbic acid, 6-epi-cucurbic acid, 6-epi-cucurbic acid-lactone, 12-hydroxy-jasmonic acid, 12-hydroxy-jasmonic acid-lactone, 11-hydroxy-jasmonic acid, 8-hydroxy-jasmonic acid, tuberonic acid, tuberonic acid-O-β-glucopyranoside, cucurbic acid-O-β-glucopyranoside, 5,6-didehydro-jasmonic acid, 6,7-didehydro-jasmonic acid, 7,8-didehydro-jasmonic acid and a lower alkyl ester thereof and a nanocarrier or microcarrier, wherein the active principle is included into, inside, associated with, conjugated with, and/or at the nanocarrier or microcarrier, and the nanocarrier or microcarrier comprises a liposome selected from a PEGylated liposome, poly(ethylene glycol)-600-hydroxystearate (PEG-HS), and LDE, the liposome being a unilamellar vesicle or a multilamellar vesicle, a dendrimer, linear bis poly(ethylene glycol) (PEG) polymer with branched spacers, a hyaluronan polymer, a peptide nanovesicle, a lipid nanotube, a cationic solid lipid microparticle, a gelatin nanoparticle, a polylactic glycolic acid nanoparticle, a hydrogel microparticle, chitosan, or a lipoglycoprotein, and further wherein the pharmaceutical formulation is in the form of an emulsion.

2. The pharmaceutical formulation of claim 1, wherein the active principle has a concentration of no greater than 1 M.

3. The pharmaceutical formulation of claim 1, wherein the active principle and the liposome form an inclusion complex.

4. The pharmaceutical formulation of claim 1, wherein the active principle is methyl jasmonate or jasmonic acid.

5. The pharmaceutical formulation of claim 1, wherein the nanocarrier or microcarrier is biodegradable.

6. The pharmaceutical formulation of claim 1, wherein the nanocarrier or microcarrier comprises linear bis poly(ethylene glycol) (PEG) polymer with branched spacers, a hyaluronan polymer, a peptide nanovesicle, a lipid nanotube, a cationic solid lipid microparticle, a gelatin nanoparticle, a polylactic glycolic acid nanoparticle, a hydrogel microparticle, chitosan, a lipoglycoprotein,

7. A method for treating cancer, comprising administering to a subject in need thereof a therapeutically effective amount of the pharmaceutical formulation of claim 1.

8. A method for treating a virus, bacterial, or fungal disease, comprising administering to a subject in need thereof a therapeutically effective amount of the pharmaceutical formulation of claim 1.

9. A method for treating an inflammatory disease, comprising administering to a subject in need thereof a therapeutically effective amount of the pharmaceutical formulation of claim 1.

10. A method for treating an autoimmune disease, dystrophies, or depression, comprising administering to a subject in need thereof a therapeutically effective amount of the pharmaceutical formulation of claim 1.

11. A cosmetic formulation comprising an active principle selected from methyl jasmonate, jasmonic acid, 7-iso-jasmonic acid, 2,3-didehydrojasmonic acid, 3,4-didehydrojasmonic acid, 3,7-didehydrojasmonic acid, 4,5-didehydrojasmonic acid, 4,5-didehydro-7-isojasmonic acid, cucurbic acid, 6-epi-cucurbic acid, 6-epi-cucurbic acid-lactone, 12-hydroxy-jasmonic acid, 12-hydroxy-jasmonic acid-lactone, 11-hydroxy-jasmonic acid, 8-hydroxy-jasmonic acid, tuberonic acid, tuberonic acid-O-β-glucopyranoside, cucurbic acid-O-β-glucopyranoside, 5,6-didehydro-jasmonic acid, 6,7-didehydro-jasmonic acid, 7,8-didehydro-jasmonic acid and a lower alkyl ester thereof, and a nanocarrier or microcarrier, wherein the active principle is included into, inside, associated with, conjugated with, and/or at the nanocarrier or microcarrier, and the nanocarrier or microcarrier comprises a liposome selected from a PEGylated liposome, poly(ethylene glycol)-600-hydroxystearate (PEG-HS), and LDE, the liposome being a unilamellar vesicle or a multilamellar vesicle, a dendrimer, linear bis poly(ethylene glycol) (PEG) polymer with branched spacers, a hyaluronan polymer, a peptide nanovesicle, a lipid nanotube, a cationic solid lipid microparticle, a gelatin nanoparticle, a polylactic glycolic acid nanoparticle, a hydrogel microparticle, or a lipoglycoprotein, and further wherein the cosmetic formulation is in the form of an emulsion.

12. The cosmetic formulation of claim 11, wherein the active principle is methyl jasmonate or jasmonic acid.

13. The cosmetic formulation of claim 11, wherein the nanocarrier or microcarrier comprises a dendrimer selected from polyamidoamine (PAMAM) G4 hydroxyl terminated dendrimer, Methotrexate-carrying PAMAM (PAMAM/MTX), and Methotrexate-carrying PEGylated PAMAM (PAMAM-PEG/MTX).
